(19) United States
(12) Patent Application Publication
(10) Pub. No.: US 2025/0261682 A1
(43) Pub. Date: Aug. 21, 2025

(54) METHOD AND PLANT FOR EXTRACTING NICOTINE FROM TOBACCO

(71) Applicant: COMAS - COSTRUZIONI MACCHINE SPECIALI - S.P.A., Silea (Treviso) (IT)

(72) Inventors: Gianfranco GRANZOTTO, Silea (Treviso) (IT); Luca NADERLOTTO, Silea (Treviso) (IT); Paolo PALLADO, Silea (Treviso) (IT)

(21) Appl. No.: 18/857,374

(22) PCT Filed: Apr. 21, 2023
(86) PCT No.: PCT/IB2023/054096

§ 371 Date: Oct. 16, 2024

(30) Foreign Application Priority Data
Apr. 28, 2022 (IT) 102022000008405

(51) Int. Cl. A24B 15/24; B01D 3/14; B01D 5/00; C07D 401/04

(52) U.S. Cl. CPC A24B 15/243; B01D 3/148; B01D 5/006; C07D 401/04

(57) ABSTRACT

Plant for the production of nicotine in the liquid state starting from tobacco-based starting material characterized in that it includes:
a macerator configured to mix said starting material with an aqueous solution, and form a first liquid fraction, which includes a mixture of said aqueous solution and nicotine, and a first solid fraction, which contains the scraps of said starting material,
a filter configured to separate said first liquid fraction from said first solid fraction,
a first heat exchanger configured to heat, and preferably to overheat said first liquid fraction and to work at a pressure higher than the ambient one, and preferably at a pressure between 0.5 barg and 3 barg, and more preferably at about 2 barg.

The present invention relates to a method and a plant for extracting nicotine from tobacco.

Methods and systems are known for extracting nicotine from tobacco, its by-products and processing waste (ribs, leaf fragments, dust, etc.).

One of these known methods involves extracting nicotine from tobacco by-products and processing waste using traditional organic solvents. This is an extraction method that is effective in itself, which however has some drawbacks, as it requires the use of toxic solvents, such as dichloromethane, alcohol, chloroform, or ethers. The solvent is then evaporated to recover the nicotine, which always contains traces of non-evaporated solvent. Therefore this method is not suitable for the extraction of nicotine destined to be used in liquids for electronic cigarettes, and therefore destined to enter the lungs of smokers, as it could be unacceptably toxic. Furthermore, extraction with organic solvents is not very selective, as it also extracts terpenes and other alkaloids present in the starting material and then requires a purification step to obtain nicotine of high purity and/or concentration.

Another known method involves extracting the nicotine using supercritical CO₂. It is a technology that does not require the use of toxic solvents and which therefore does not present the aforementioned drawback but, on the other hand, requires working at a pressure higher than 200 bar and involves the need to use materials and structures resistant to these pressures with production batches modest in quantity. It follows that the extraction of nicotine with this method is a rather expensive process. Furthermore, extraction with CO₂ is not selective and, while being very effective in removing aromas and terpenes, it is not particularly suitable for the extraction of nicotine alone.

Another known method provides for the nicotine extraction to be by concentrating a mixture of water laden with nicotine by successive stages of evaporation carried out at low pressure, lower than ambient pressure. It is a method which, similarly to the previous one, does not require the use of toxic solvents but involves evaporating the water from a mixture containing nicotine. This requires long execution times and a large amount of energy, since the evaporation must be carried out at very low pressure and at a high temperature of the mixture in order to allow effective evaporation of the water. Furthermore, since water and nicotine are not miscible each other with at all temperatures and at all concentrations and tend to form a low-boiling azeotrope, this known method alone does not allow to obtain nicotine with the degree of purity required for its uses and requires the development of a subsequent purification step to obtain the desired degree of purity.

The object of the invention is to propose a method and a plant which allow nicotine to be extracted from tobacco by-products and processing waste.

Another object of the invention is to propose a nicotine extraction method and plant which allow to overcome the drawbacks that the known extraction methods involve.

Another object of the invention is to propose a method and a plant which do not require the use of toxic solvents.

Another object of the invention is to propose a method and a plant which do not require the use of equipment capable of withstanding high temperatures and high pressures.

Another object of the invention is to propose a method and a plant which do not require high energy consumption.

Another object of the invention is to propose a method and a plant which can be implemented with limited costs and which can ensure high production potential.

Another object of the invention is to propose a method and a plant which allow to extract nicotine with a very high degree of purity.

Another object of the invention is to propose a method and a plant which allow to reduce the waste of material.

Another object of the invention is to propose a method and a plant which allow to obtain high yields in the extraction of nicotine from the starting material. Another object of the invention is to propose a method and a plant which allow energy to be efficiently used.

Another object of the invention is to propose a method and a plant which exploit the chemical properties of the starting materials.

Another object of the invention is to propose a method and a plant which can be operated/implemented with maximum safety for the operators.

Another object of the invention is to propose a method and a plant which can be used to produce large quantities of nicotine.

Another object of the invention is to propose a method and a plant which can be used with dangerous substances even in small quantities.

Another object of the invention is to propose a method and a plant which reduce the overall dimensions to a minimum.

Another object of the invention is to propose a method and a plant which are alternative and/or better than the known ones.

Another object of the invention is to propose a method and a plant which can be implemented/carried out easily and with low costs.

All these objects and others which will result from the following description are achieved according to the invention with a plant for the extraction of nicotine from tobacco as defined in claim 1, with a method as defined in claim 8 and with a solution/mixture as defined in claim 15.

The present invention is hereinafter further clarified in some of its preferred embodiments shown for purely exemplifying and non-limiting purposes with reference to the attached drawings, in which:

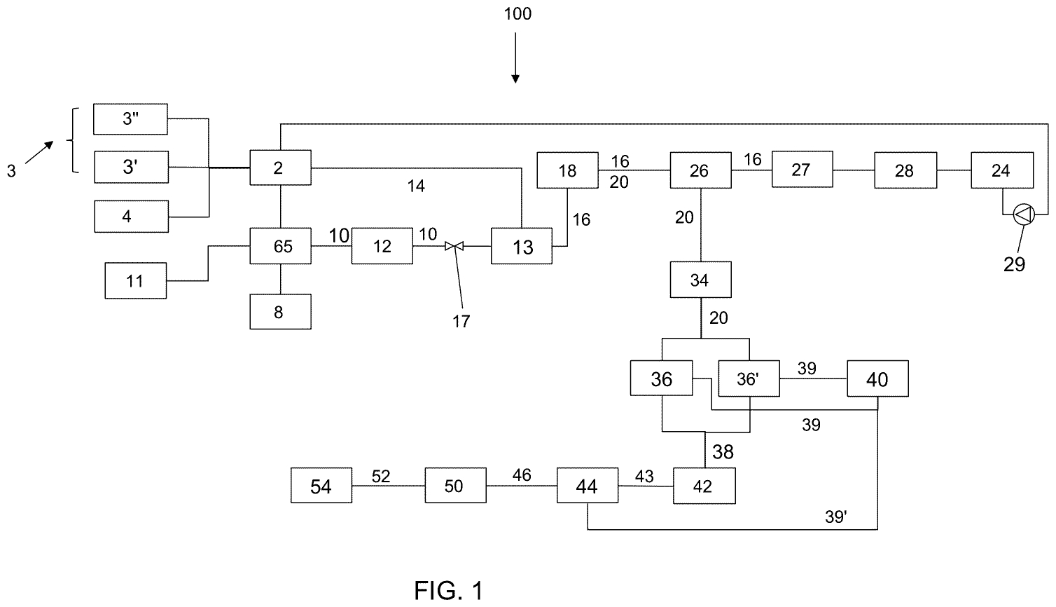

FIG. 1 is a schematic view of a plant according to the invention,

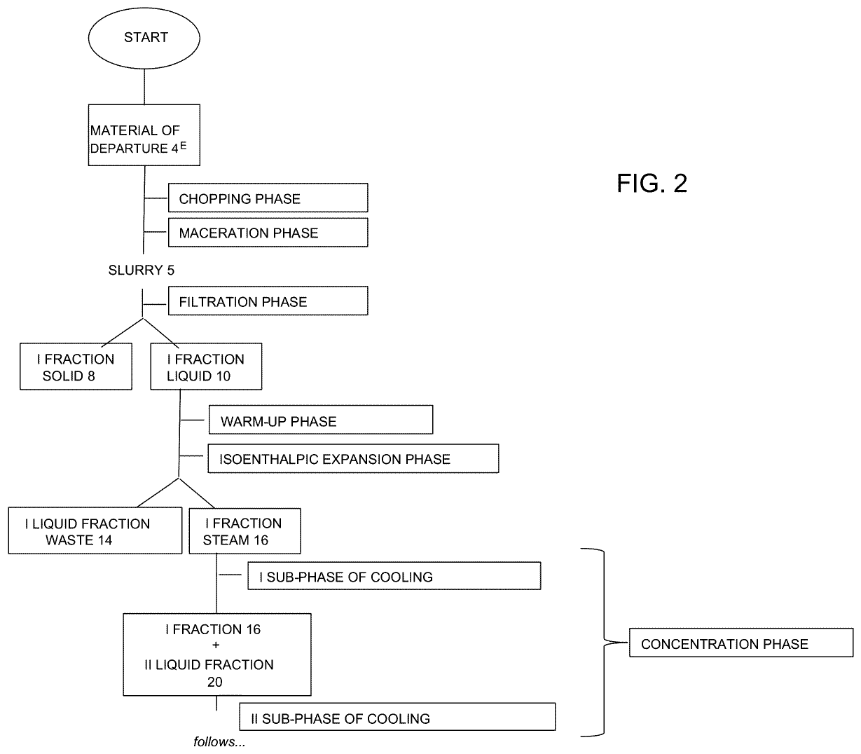

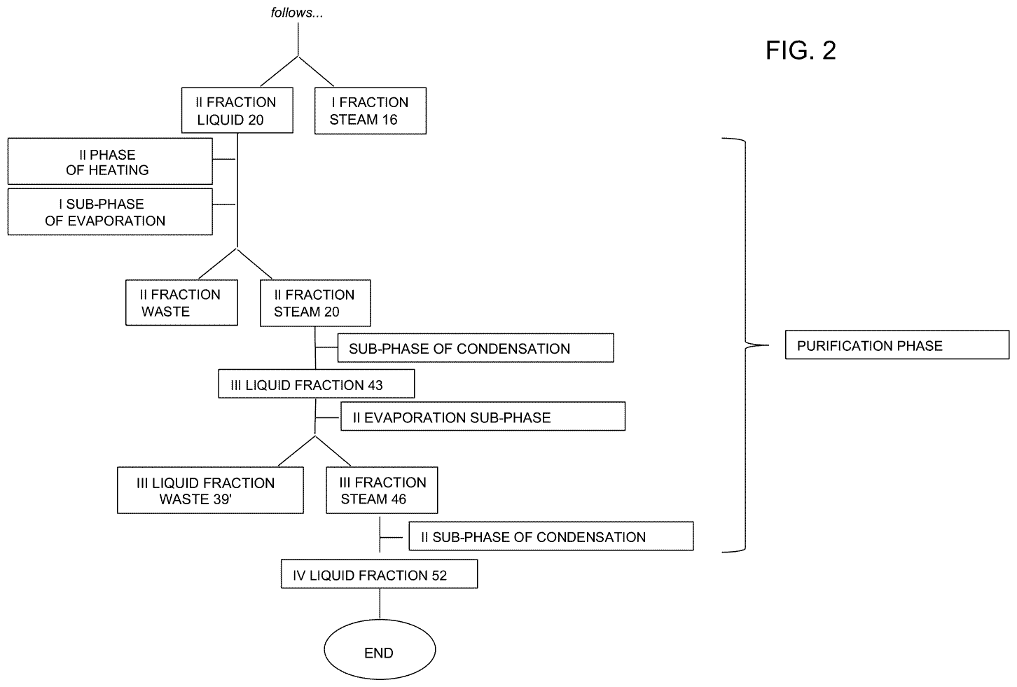

FIG. 2 shows a schematic view of a flow diagram of the method according to the invention.

The method and plant according to the invention are used to treat a starting material, which preferably comprises tobacco by-products and tobacco processing waste (ribs, leaf fragments, dust, etc.), but could also comprise whole tobacco. Conveniently, the starting material can have a nicotine concentration ranging from about 0.7% to about 6%, and generally equal to about 2%; here and below, unless otherwise indicated, the percentages are always to be understood as expressed by weight.

Further, the starting material may have a water content of from about 2% to about 15%, and is generally about 6%.

The plant 100 according to the invention can comprise shredding means, (not shown since they are traditional in themselves), which make it possible to reduce the size of the starting material 4 to dimensions useful for subsequent treatments and generally between 0.1 mm and 20 mm, preferably between 0.2 mm and 5 mm.

Conveniently the comminution means can comprise a shredder or a grinder or the like. In one embodiment there may be a plurality of shredders arranged in series, in order to reduce the starting material 4 to a suitable size.

A macerator 2 is provided downstream of the shredding means, i.e. a tank into which the starting material 4 is introduced, brought to the substantially uniform dimensions previously indicated, and other substances suitable for subjecting said starting material to a maceration treatment in order to obtain a mixture 5 or “slurry”. Preferably these substances consist of an aqueous solution 3, which for example can comprise water 3′ and sodium hydroxide 3″.

Since the maceration process requires that the mixture comprising the starting material 4 and the aqueous solution 3 be kept in constant stirring, it is foreseen that the macerator 2 is provided with suitable means for creating this mixture and these can be constituted for example by a mechanical agitator, with which the macerator 2 is internally provided, or by a fluid current generated by a mechanical vortex induced by the thrust of a circulation pump external to the macerator, or by the suction generated by a Venturi tube fed with a circulated fluid inside the macerator by means of an external pump. In the case of a mechanical agitator, this can consist of a rotating agitator of the blade or propeller or turbine type, and preferably a blade having a rotation speed generally between 10 rpm and 1000 rpm, and preferably about 100 rpm. Instead, in the case of an external pump, this can advantageously be single-screw, or peristaltic, or centrifugal or with a backward impeller and preferably a centrifugal pump with a backward impeller.

Furthermore, the macerator 2 can be provided with suitable heating means suitable for heating and/or maintaining the mixture present inside it at a temperature higher than the ambient temperature.

The macerator 2, which transforms the products introduced into it into the “slurry” 5, is connected to a mechanical filter 6, configured to separate from the “slurry” 5 a solid fraction 8, possibly to be inerted, and a first liquid fraction 10, which substantially comprises, and preferably consists of a liquid mixture containing the aqueous solution 3, nicotine and other components solubilized in the maceration step.

More generally, the filter 6 can be of various types: a filter press, a horizontal centrifuge, a simple filter cloth or one previously treated with a coating of diatomaceous earth, a bell filter or the like. In general, however, a filter press or a horizontal centrifuge are preferable. In the specific case of the horizontal centrifuge, this is preferable when the slurry lacks a fibrous part, for example when an aggressive chemical agent such as caustic soda is used.

The solid fraction 8 can conveniently be subsequently disposed of, preferably after a neutralization process aimed at bringing its pH back to a substantially neutral value by adding an acid solution 11.

In the illustrated embodiment the acid solution 11 is added directly inside the filter 6. It can be prepared starting from a concentrated solution having a concentration between about 20% and 50%, and preferably about 35%, which it is diluted to bring it to a concentration of between 0.5% and 10%, generally 5%, before being fed to the filter 6 to come into intimate contact with the solid fraction 8. Alternatively, in an embodiment not shown in the drawings, the acid solution 11 can be diluted inside the macerator 2.

Advantageously, the plant 100 also comprises a first heat exchanger 12, configured to receive the first liquid fraction 10 and to heat it. Preferably, the first heat exchanger 12 can be configured to maintain the first liquid fraction 10 at a pressure of between 2.5 barg and 5 barg and at a temperature of between 125° C. and 150° C., and preferably around 140° C. Conveniently, the first heat exchanger 12 can be configured to maintain the first liquid fraction 10 at a pressure ranging from 3.5 barg to 5 barg and at a temperature ranging from 105° C. to 140° C., and preferably around 120° C.

With “barg” (or bar gauge) it is intended the unit of measurement representing the difference between the pressure in bar in a space and the atmospheric pressure in bar.

Advantageously, the first heat exchanger 12 can be configured to advance the first liquid fraction 10, in particular by means of thrust means such as for example a pump (not shown).

Preferably, the first heat exchanger 12 can be configured to maintain the first liquid fraction 10 substantially in the liquid state, i.e. without allowing its even partial evaporation. In particular therefore the first heat exchanger 12 can comprise an internal cavity which can be completely filled by the first liquid fraction 10 and can therefore prevent its evaporation.

In a different embodiment, not shown, the first heat exchanger 12 can be replaced by a plurality of first heat exchangers, preferably arranged in series and configured to heat and preferably superheat the first liquid fraction 10 up to the desired temperature, advantageously without causing even partial evaporation.

Suitably the first heat exchanger 12 is fluidically connected to a first evaporation chamber 13 configured to allow the first liquid fraction 10 to separate into a first waste liquid fraction 14 and a first vapor fraction 16. Suitably in the fluid connection between the first heat exchanger 12 and the first evaporation chamber 13 a lamination valve 17 can be provided.

The first evaporation chamber 13 can advantageously be configured to receive the first superheated liquid fraction 10 and allow its isoenthalpic expansion, and can be fluidically connected to a second heat exchanger 18. Conveniently this fluidic connection can be positioned at the top of the first evaporation chamber 13, so that preferably only the first vapor fraction 16 can pass through it.

Advantageously, the first evaporation chamber 13 can also be fluidically connected to the macerator 2 and this connection can advantageously be positioned at the bottom, and preferably at the bottom of the first evaporation chamber 13, so that preferably only the first waste liquid fraction 14 may pass.

Advantageously, the second heat exchanger 18 can be configured to allow at least partial cooling of the first vapor fraction 16 and to thus obtain its partial condensation, in order to form a second liquid fraction 20 in equilibrium with the first vapor fraction 16. In other words, the exchanger 18 can be used to maintain a phase equilibrium, preferentially condensing the nicotine and keeping the water as much as possible in the vapor phase. Conveniently the second heat exchanger 18 can be configured to bring and/or maintain the second liquid fraction 20 and the first vapor fraction 16 at a temperature of about 100° C. and to operate at positive pressure, and preferably at a pressure of about 1 barg.

The second heat exchanger 18 can be fluidically connected with a third heat exchanger 26 configured to further cool said first vapor fraction 16 and said second liquid fraction 20. In particular, the third heat exchanger 26 can be configured for cooling the second liquid fraction 20 and/or the first vapor fraction 16 to a temperature lower than 50° C., for example to a temperature of about 32° C.-45° C., and more preferably to a temperature of 38° C.-42° C.

Advantageously, the third heat exchanger 26 can be configured to operate at negative relative pressure, i.e. at a pressure lower than atmospheric pressure, in particular at a pressure of about 0.05 bar.

Conveniently the fluidic connection between the second heat exchanger 18 and the third heat exchanger 26 can be made in a slight descent, for example inclined by about 5%, and originate at the lower part, and preferably at the bottom of the second exchanger of heat 18, so that the second liquid fraction 20 can flow substantially by gravity in the direction of the third heat exchanger 26.

Conveniently, the third heat exchanger 26 can be fluidically connected to a tank 24 for recovering the first vapor fraction 16. Conveniently, between the third heat exchanger 26 and the tank 24, a fourth heat exchanger 27 can be positioned, configured to cool and condense the vapor first fraction 16. In particular, the fourth heat exchanger 27 can be configured to cool the vapor first fraction 16 to a temperature of about 25° C.

In an alternative embodiment, if the first vapor fraction 16 comprises a relevant nicotine concentration, for example around 0.1%, the fourth heat exchanger 27 can be configured to supply heat to the first vapor fraction 16 in order to favor total evaporation of the water contained in the first vapor fraction 16 and to allow at least partial segregation of the nicotine transported by the first vapor phase.

Conveniently, furthermore, a fifth heat exchanger 28 may be present, configured to cool the first vapor fraction 16, and also allow the residual vapor phase to condense.

In order to send the first vapor fraction 16 towards the tank 24, a vacuum production apparatus 29 can advantageously be provided, for example comprising a suction pump. This can be arranged downstream of any of the exchangers 26, 27 and 28 or, as shown in FIG. 1, downstream of tank 24.

The vacuum production apparatus can also be suitably fluidically connected with the second heat exchanger 18 and with the first evaporation chamber 13, so as to allow the flow of the first vapor fraction 16 along its path. Thanks to the connections existing between the tank 24, the fifth heat exchanger 28, the fourth heat exchanger 27, the third heat exchanger 26, the second heat exchanger 18 and the first evaporation chamber 13, all these components can be sucked from the vacuum production apparatus 29.

Advantageously, the third heat exchanger 26 can be fluidically connected to at least one sixth heat exchanger 34 configured to heat the second liquid fraction 20 to a temperature higher than 50° C. and preferably to a temperature of about 70° C. The sixth heat exchanger 34 can also be fluidically connected to the vacuum production apparatus 29, which therefore allows the extraction of the water vapor that can form, and which can therefore be added to the first vapor fraction 16.

The sixth heat exchanger 34 can be fluidically connected with at least one second evaporation chamber 36, and preferably with a pair of second evaporation chambers 36, 36′ configured to obtain from the second liquid fraction 20 a second vapor fraction 38, which substantially contains almost exclusively nicotine, and a second waste liquid fraction 39, which for example can comprise alkaloids contained in the starting material 4 and different from nicotine.

The desirability of having a pair of second evaporation chambers 36, 36′ instead of a single second evaporation chamber 36 will become apparent hereinafter.

Advantageously, if several evaporation chambers 36, 36′ are present, these can be substantially identical to each other.

Conveniently, the fluid connection between the sixth heat exchanger 34 and the second evaporation chambers 36, 36′ can originate at the bottom of the sixth heat exchanger 34, so as to allow the second liquid fraction 20 to flow by gravity towards the second evaporation chambers 36, 36′.

Conveniently there may be a flow diverter configured to direct the flow of the second liquid fraction 20 towards only one of the second evaporation chambers 36, 36′.

The second evaporation chambers 36, 36′ are suitably configured to allow the evaporation of the second vapor fraction 38 which substantially contains pure nicotine in the vapor state. In particular, therefore, the second evaporation chambers 36, 36′ can comprise suitable heating means, configured to carry out the heating of the second liquid fraction 20 and its consequent evaporation.

Conveniently, the second evaporation chambers 36, 36′ can be fluidically connected with suction means, which allow the second vapor fraction 38 to be sucked.

Advantageously, at the lower portion of each second evaporation chamber 36, 36′ there may be a connecting duct with a container 40 for collecting the second waste liquid fraction 39.

Suitably the second evaporation chambers 36, 36′ can be connected to a seventh heat exchanger 42, configured to bring the second vapor fraction 38 to the liquid state and therefore to obtain a third liquid fraction 43. Therefore the seventh heat exchanger 42 can be configured to cool the second vapor fraction 38.

Conveniently, the seventh heat exchanger 42 is fluidically connected to a third evaporation chamber 44 substantially similar to the second evaporation chambers 36, 36′. In particular, the third evaporation chamber 44 can also be configured to obtain from the third liquid fraction 43 a third vapor fraction 46 and a third waste liquid fraction 39′.

Advantageously, at the bottom of the third evaporation chamber 44 there may be a discharge duct connected to the container 40 for collecting the highboiling liquid products.

Conveniently, the third evaporation chamber 44 can comprise suitable heating means configured to heat the third liquid fraction 43 and to consequently cause its evaporation.

Conveniently, the third evaporation chamber 44 can be fluidically connected to suction means, which suck in the third vapor fraction 46.

Advantageously, the third vapor fraction 46 can be made to pass through an eighth heat exchanger 50 configured to cool the third vapor fraction 46 and to cause its condensation in order to obtain a fourth liquid fraction 5, which. can advantageously be collected inside a tank 54.

The present invention also relates to a method for producing liquid nicotine starting from a tobacco-based starting material.

The method according to the invention preferably provides for the use of an implant 100 as described above.

The starting material 4 can conveniently be ground up beforehand in order to have a size suitable for carrying out the method described herein, and generally between 0.1 mm and 20 mm, preferably between 0.2 mm and 5 mm.

The method conveniently provides for a first phase of maceration of the starting material 4 which has been previously comminuted, in order to bring at least a portion of the nicotine contained therein into solution. In particular, said maceration step can take place inside the macerator 2. This maceration step can be carried out in an aqueous solvent 3′, preferably at basic pH, in order to facilitate the maceration of the starting material. Conveniently, the desired pH can be obtained by adding to the solvent a predefined quantity of a 3″ base, preferably a strong base, for example sodium hydroxide (NaOH). In one embodiment, the aqueous solution 3 can be introduced into the macerator 2 at the concentration—i.e. at the pH—desired. Alternatively, a more concentrated solution, for example at a concentration between about 20% and 50%, and preferably about 33%, can be prepared beforehand, and subsequently introduced into the macerator 2, inside which it is already water present. The sodium hydroxide 3″ can also be added in the solid state directly inside the macerator 2. In this way the dissolution reaction of the sodium hydroxide and/or the process of increasing the concentration of sodium hydroxide in the solvent present inside the macerator 2, both exothermic processes, help to increase the temperature of the mixture during the maceration phase, thus facilitating the extraction of the nicotine. Advantageously, sodium hydroxide can be present in about 5%-20%, and preferably in about 10% with respect to the aqueous solution.

Advantageously from 4 m³ to 12 m³ of aqueous solution 3 can be introduced inside the macerator 2, preferably about 7 m³ of aqueous solution for each ton of starting material 4.

The slurry 5 at the exit from the macerator 2 can have a low viscosity, and comprise a residual fraction of solid variable between 10% and 30%, generally 20% by weight, depending on the quality of the starting material 4, its size, the concentration of caustic soda, the maceration time, the maceration temperature and the tobacco/water dilution ratio.

Conveniently, during the maceration step, the slurry 5 formed by the aqueous solution 3 and the starting material 4 can be kept at a high temperature with respect to the ambient temperature in order to accelerate and make the extraction process more effective. For example, the slurry 5 can be maintained at a temperature between about 35° C. and 80° C., and preferably equal to about 50° C.

Advantageously said maceration step can last from 4 to 36 hours, depending on the size of the starting material 4, and can preferably last about 24 hours.

After the maceration phase, the process can include a filtration phase, preferably mechanical; it provides for the separation of the first liquid fraction 10, which substantially consists of the liquid mixture of aqueous solution and nicotine, from the solid fraction 8 of the starting material 4.

The solid fraction 8 can then be recovered, possibly rendered inert by the addition of an acid solution 11, and subsequently discarded and/or reused.

The acid solution can be prepared similarly to the NaOH solution, which is added in the macerator 2. The amount of acid is calculated with respect to the residual amount of soda trapped in the waste product and/or its pH, in order to arrive at a value pH between 8 and 12, preferably 9.

For the preparation of the diluted acid solution starting from a concentrated solution, which for example can be at a concentration between 20% and 50%, generally 35%, the same maceration tank 2 can be used, previously emptied of its contents.

When neutralization is complete, a rinsing phase is carried out with pure water to wash the residual acid and avoid direct contact between it and the caustic solution.

Conveniently, the first liquid fraction 10 can be subjected to a first heating phase in the first heat exchanger 12, and in particular to a first superheating phase, during which it is brought to a high temperature, preferably to a temperature between 105 and 140° C., and preferably around 120° C.

Conveniently during said first heating step any evaporation of the first liquid fraction 10 is avoided, for example by carrying out the first heating step inside a chamber, the volume of which is completely occupied by the first liquid fraction itself. The first heating step can conveniently be carried out by maintaining the first liquid fraction 10 at a high pressure, for example at a pressure ranging from 3.5 to 5 barg.

Subsequently, the first liquid fraction 10 thus overheated can be subjected to an isoenthalpic expansion step. Conveniently, for this purpose it can be made to pass through the lamination valve 17 to then enter the first evaporation chamber 13. Conveniently during this phase the first liquid fraction 10 separates into a first vapor fraction 16 and a first liquid fraction of waste 14.

In particular, the first vapor fraction 16 can have a nicotine concentration equal to the azeotropic concentration, i.e. equal to about 2.5%, but it can also contain substantially all the nicotine that was present in the first liquid fraction 10. It can be recovered to be subjected to further steps of concentration and/or purification. In particular, it can be subjected to a shock condensation phase aimed at removing most of the water still present.

The first steam fraction 16 can then be subjected in the second exchanger 18 to a first sub-phase of cooling at a positive relative pressure, for example at a pressure of about 1 barg. Conveniently, during the first sub-phase of cooling, the first vapor fraction can be brought to a temperature of about 100° C.-120° C. Advantageously, during the first cooling sub-phase, a portion of the first vapor fraction 16 can condense to form a second liquid fraction 20. Therefore, since nicotine has a higher boiling point than water, the second liquid fraction 20 is more rich in nicotine.

The first liquid waste fraction 14 can instead be sent to the macerator 2 in order to be reused, even several times, in particular up to three times, to recover any nicotine residues present therein.

Subsequently, the part of the first vapor fraction 16 not yet condensed and the second liquid fraction 20 leaving the second exchanger 18 can be subjected to a second sub-phase of cooling in the third heat exchanger 26. This can be carried out at negative relative pressure, for example at about 50 mbar and leads to a further condensation of the first vapor fraction 16, and therefore to an increase of the second liquid fraction 20. Conveniently, during the second cooling sub-phase the second liquid fraction 20 and the first vapor fraction 16 can be cooled down to a temperature lower than 50° C., for example to a temperature of about 32° C.-45° C., and preferably to a temperature of 38° C.-42° C.

Advantageously, the reduction of the pressure during the second cooling sub-phase leads to a further evaporation of the lowest-boiling component of the second liquid fraction 20, i.e. water, thus contributing to a concentration of the nicotine inside the liquid fraction itself.

If the first cooling sub-phase has been carried out in the second heat exchanger 18, the second liquid fraction 20 can flow substantially by gravity from the second heat exchanger 18 to the third heat exchanger 26.

Subsequently the first vapor fraction 16, which is not condensed in the second liquid fraction 20 and which substantially comprises only water, is aspirated and sent to a tank 24 to be recovered and sent back to the macerator 2. Preferably before being sent back to the macerator 2 the first vapor fraction 16, which is not condensed in the second liquid fraction 20, can be subjected to a further condensation step, for example in the fourth heat exchanger 27 and/or in the fifth heat exchanger 28 upstream of the tank 24.

Furthermore, the second liquid fraction 20 can be subjected to a second heating phase, for example inside the sixth heat exchanger 34, in order to cause the evaporation of any residual water present therein. Preferably, for this purpose, the second liquid fraction 20 can be taken from the bottom of the third heat exchanger 26 and sent, through a suitable duct, to the sixth heat exchanger 34.

In particular, the second heating phase can be carried out at low pressure, to allow the evaporation of any last residues of water, which are then sucked up by the vacuum production apparatus 29 together with the part of the first steam fraction 16 not yet condensed. Advantageously, the water vapor thus generated can rise, guided by the depression generated by the vacuum production apparatus 29, along the same duct along which the second liquid fraction 20 descends.

In the sixth heat exchanger 34 the second liquid fraction 20 is subjected to at least one purification step aimed at separating the nicotine from any other compounds which were present within the starting material 4 and which have been extracted from the second liquid fraction 20. For example, the purification step can be aimed at separating nicotine from other alkaloids with a higher boiling point. In particular, the purification step can include at least one distillation sub-step, and preferably two distillation sub-steps. Conveniently, for this purpose the second liquid fraction 20 can be sent to the second evaporation chamber 36, 36′. Advantageously, during the first evaporation sub-phase, the second liquid fraction 20 can be heated in order to separate from it a second vapor fraction 38, which substantially contains almost only nicotine, and a second waste liquid fraction 39, which substantially contains only alkaloids with a boiling point higher than that of nicotine and which is collected inside the container 40.

If several evaporation chambers 36, 36′ are present, the process can proceed alternately, and, in particular, in a chamber 36, which has been isolated with respect to the upstream portion of the plant 100, in order to avoid reflux of the second vapor fraction 38, and was connected to the downstream portion of the plant in order to allow the second vapor fraction 38 to continue towards the further steps and/or sub-steps of the method according to the invention and/or towards the components of the plant positioned downstream, the distillation process takes place, in which the second liquid fraction 20 is heated until it evaporates, at least partially. At the same time the other chamber 36′ is connected both with the container 40, in order to discharge the second waste liquid fraction 39′, and with the portion upstream of the plant 100, in order to continue collecting the second fraction liquid 20 that is produced.

The second vapor fraction 38, which leaves the evaporation chambers 36, 36′, is then subjected in the seventh heat exchanger 42 to a condensation sub-phase, in order to be returned to the liquid phase, thus forming the third liquid fraction 43.

Subsequently, the third liquid fraction 43 can be subjected in the third evaporation chamber 44 to a second distillation sub-phase, substantially similar to the previous one, in which it is separated into a third waste liquid fraction 39′, and a third vapor fraction 46, which contains almost exclusively nicotine, for example in a concentration between 92% and 99.7%, generally 99.5%.

Subsequently, the third vapor fraction 46 can be subjected in the eighth heat exchanger 50 to a second condensation sub-phase, which generates the fourth liquid fraction 52 intended to be collected inside the tank 54.

According to an alternative not shown, the purification phase can take place in a fractional manner by using a temperature probe to be applied before the condensation phases which allow the recovery of the nicotine, and a deviation line which, by means of a shut-off valve is able to intercept the volatile compounds at the beginning of the distillation (and only subsequently the heavy components), preferentially recovering the nicotine.

From what has been said it is clear that the method and plant according to the invention are particularly advantageous, indeed optimal, because:
allow to obtain a very pure and/or concentrated final product,
allow you to optimally reuse the starting material by reducing waste,
allow you to use energy efficiently, reducing consumption.

1. Plant for the production of nicotine in the liquid state starting from tobacco-based starting material characterized in that it comprises:
a macerator configured to mix said starting material with an aqueous solution, and form a first liquid fraction, which comprises a mixture of said aqueous solution and nicotine, and a first solid fraction, which contains the scraps of said starting material,
a filter configured to separate said first liquid fraction from said first solid fraction,
a first heat exchanger configured to heat, and preferably to overheat said first liquid fraction and to work at a pressure higher than the ambient one, and preferably at a pressure between 0.5 barg and 3 barg, and more preferably at about 2 barg.

2. Plant according to claim 1 wherein said first heat exchanger is fluidically connected to a first evaporation chamber in which said first liquid fraction is separated into a second liquid fraction and then a first steam fraction, and that said fluidic connection comprises a throttling valve.

3. Plant according to claim 2 wherein it comprises a second heat exchanger fluidically connected, preferably directly, with said first evaporation chamber and configured to cool said first vapor fraction in order to obtain its condensation, at least partially, in a second liquid fraction.

4. Plant according to claim 3 wherein it comprises a third heat exchanger fluidically connected, preferably directly, with said second heat exchanger and associated with cooling means for cooling said first steam fraction and/or said second liquid fraction.

5. Plant according to claim 3 wherein said second exchanger and/or said third exchanger are fluidically connected with said macerator in order to send back into the latter, for its recondensation, said first steam fraction, preferably after its passage through a fourth heat exchanger.

6. Plant according to claim 3 wherein it comprises at least one second evaporation chamber, and preferably at least two second evaporation chambers fluidly connected with said second exchanger and/or with said third exchanger.

7. Plant according to claim 3 wherein it comprises at least two second evaporation chambers and a flow diverter configured to send said second liquid fraction alternately to one or the other second evaporation chamber.

8. Method for the production of nicotine in the liquid state starting from tobacco-based starting material wherein it comprises:
a phase of maceration of the starting material in an aqueous solution, to obtain a pulp or slurry,
a filtration step of said mixture to separate a first liquid fraction from a first solid fraction,
a step of superheating said first liquid fraction carried out at a pressure higher than atmospheric pressure, and preferably at a pressure ranging from 0.5 barg to 3 barg, and more preferably at about 2 barg.

9. Method according to claim 8 wherein in said maceration step a strong base is used in a concentration preferably between 20% and 50%, and more preferably equal to 33% by weight.

10. Method according to claim 8 wherein it comprises an isoenthalpic expansion phase of said first superheated liquid fraction in order to separate a first vapor fraction containing a concentration of nicotine preferably equal to the azeotropic concentration, and a first waste liquid fraction.

11. Method according to claim 10 wherein said isoenthalpic expansion phase is followed by a first condensation phase of said first vapor fraction which leads to the formation of a second liquid fraction, which is then subjected to at least one phase concentration aimed at removing water vapor from the second liquid fraction itself, said concentration step preferably comprising:
a first sub-step of cooling said second liquid fraction at positive relative pressure, preferably at a pressure of about 1 barg, and at a temperature between about 100° C.-120° C., and
a second cooling sub-step at negative gauge pressure, and preferably at an absolute pressure of about 50 mbar, and at a temperature below 50° C., preferably at a temperature of about 32° C.-45° C., and more preferably at a temperature of 38° C.-42°C.

12. Method according to claim 11 wherein said second liquid fraction is subjected to at least one purification step aimed at removing unwanted compounds present within it and having a boiling point higher than that of nicotine, and in that said purification step comprises at least one distillation sub-step, and preferably two distillation sub-steps.

13. Method according to claim 9 wherein said at least one distillation sub-phase is carried out in two second distillation chambers arranged in parallel, and that while in a distillation chamber the distillation process, the other is filled with the second liquid fraction and/or emptied of said unwanted compounds.

14. Method according to claim 1 wherein it is implemented with a plant.

15. Solution and/or mixture comprising nicotine and water, in which the nicotine is present in a concentration ranging from 92% to 99.7%, generally 99.5%, wherein it is obtained with a plant according to claim 1.

16. Solution and/or mixture comprising nicotine and water, in which the nicotine is present in a concentration ranging from 92% to 99.7%, generally 99.5%, wherein it is obtained with a method according to claim 8.
